(19) United States
(12) Patent Application Publication
(10) Pub. No.: US 2010/0099759 A1
(43) Pub. Date: Apr. 22, 2010

(54) AGRICULTURAL APPLICATIONS OF SILVER DIHYDROGEN CITRATE

(76) Inventors: Michael L. KRALL, El Cajon, CA (US); Andrew B. Arata, Lake City, FL (US)

Correspondence Address: MORRISON & FOERSTER LLP, 12531 HIGH BLUFF DRIVE, SUITE 100, SAN DIEGO, CA, 92130-2040, US

(21) Appl. No.: 12/480,613

(22) Filed: Jun. 8, 2009

Related U.S. Application Data
(60) Provisional application No. 61/059,334, filed on Jun. 6, 2008.
(60) Provisional application No. 61/099,291, filed on Sep. 23, 2008.

(51) Int. Cl. A61K 31/28

(52) U.S. Cl. 514/495

(57) ABSTRACT

A method for treating a plant, the method comprising contacting a composition comprising silver dihydrogen citrate with at least one of the plant, a portion of soil and/or groundwater proximal to the plant, or a combination thereof.

### CROSS-REFERENCE TO RELATED APPLICATIONS

This application claims priority to U.S. provisional patent application Ser. No. 61/059,334, filed Jun. 6, 2008, and Ser. No. 61/099,291, filed Sep. 23, 2008, and PCT patent application No. PCT/US2008/079946, filed Oct. 15, 2008, which are incorporated by reference in this application.

### TECHNICAL FIELD

This disclosure relates to compositions and methods of treating and/or preventing disease in plants.

### BACKGROUND

Plant Diseases have an enormous impact on agriculture and related industries. In the United States alone, the total economic costs of crop diseases have been estimated to be in excess of $30 billion per year. In addition to the expenses related to treatment and prevention, plant diseases can result in reductions in crop yield, quality, nutritional value, resistance to pest and other diseases, and fertility.

Plants are susceptible to a wide range of pathogens, including fungi, oomycetes, bacteria, viruses and other virus-like organisms. These groups of disease-causing organisms are each very different, representing such divergent groups of organisms as single-celled bacteria, multi-cellular fungi and subcellular viruses. In their native environments, many of these pathogens are kept in check by natural predators. However, with increasing world-wide trade, plant diseases have been introduced to areas where no predators are present, often leading to virulent and destructive epidemics in plants.

Conventional methods for the prevention and treatment of plant disease include the repeated use of costly treatment agents, such as pesticides and fungicides. In addition to the enormous added cost of their manufacture and application, many of these agents are be detrimental to the environment. With irrigation and runoff, these agents can be concentrated in waterways where they can contaminate soil and groundwater. In addition, these agents often have limited application to a specific crop and/or a specific disease, resulting in the need to maintain a costly inventory of multiple treatment agents.

An example of a pathogen which has spread throughout the major agricultural areas of the world is the fungus Phakopsora pachyrhizi which causes Asian soybean rust. Native to Asia, it was it was first found in South America in 2001, had infected crops in Louisiana by 2004 and is now found throughout much of the South and Midwest and continues to spread throughout the Western hemisphere. Rust is particularly destructive to soybeans and other edible legumes but can infect a wide variety of species where it causes brown necrotic spots on the under side of the leaves. Although certain fungicides can be effective deterrents, their cost is estimated at upwards of $50 an acre. The economic impact of Asian soybean rust is estimated to be as high as $2 billion dollars in the United States alone and equivalent amount in Brazil, for example.

Thus, there is a need for an economic, high activity, broad spectrum treatment agent for agricultural applications with low toxicity to plants and humans and a low risk of environmental pollution. These needs and other needs are satisfied by the compositions and methods of the present disclosure.

### SUMMARY

The present disclosure provides, in various aspects, compositions and methods of treating and/or preventing disease in plants, specifically compositions and methods comprising silver dihydrogen citrate (SDC).

In accordance with the purpose(s) of this disclosure, as embodied and broadly described herein, the present disclosure provides, in a first aspect, a method for treating a plant, the method comprising contacting a composition comprising silver dihydrogen citrate with a plant.

In a second aspect, the present disclosure provides a method, as described above, wherein the composition comprising silver dihydrogen citrate is contacted with at least a portion of an external surface of the plant.

In a third aspect, the present disclosure provides a method, as described above, wherein the composition comprising silver dihydrogen citrate is contacted with at least one of: a root of the plant, a portion of soil and/or groundwater proximal to the plant, or a combination thereof.

In a fourth aspect, the present disclosure provides a method, as described above, wherein the composition further comprises an acid.

In a fifth aspect, the present disclosure provides a method, as described above, wherein the composition further comprises at least one of an antifungal, antibacterial, antiviral agent, or a combination thereof.

In a sixth aspect, the present disclosure provides a method, as described above, wherein the plant is an agricultural crop, a horticultural plant, or a combination thereof.

Additional advantages of the disclosure will be set forth in part in the description that follows, and in part will be obvious from the description, or may be learned by practice of the various aspects of the disclosure. The advantages of the disclosure will be realized and attained by means of the elements and combinations particularly pointed out in the appended claims. It is to be understood that both the foregoing general description and the following detailed description are exemplary and explanatory only and are not restrictive of the disclosure, as claimed.

### DETAILED DESCRIPTION

Disease of plants is a major economic and social problem. While various treatments have been developed for some diseases, they often suffer from being narrow in effect and therefore limited to the treatment of a certain disease or being of such broad activity that they are toxic to desirable organisms. In addition, present treatments are usually expensive and the complexity of their synthesis limits their availability.

Silver dihydrogen citrate (SDC) has previously been shown to be a disinfectant that is effective against very wide range of pathogens and pests. For example, U.S. Pat. No. 6,583,176 and U.S. Patent Publication No. 20060115440, which are incorporated herein by reference in their entirety for the purpose of disclosing silver dihydrogen citrate and methods of making and using, indicate that SDC can be used to effectively kill a wide variety of species of bacteria, fungi, and viruses. These three broad types of pathogens—bacteria, fungi and virus—are extremely different from each other. Bacteria are single cells with very simple intracellular organization. Fungi, which include both yeasts and mushrooms, are the most primitive of the multi-cellular, or eukaryotic, life forms. Bacteria and fungi are thought to have diverged some 600 million years ago. Virus are not considered to be true life forms, as they cannot reproduce independently but are reliant on the reproductive mechanisms of the cells they infect.

An antimicrobial agent which is as effective against bacteria, fungi and yeast as is SDC by definition has wide ranging toxicity. Therefore, it is not predictable whether this same antimicrobial agent would be toxic or deleterious to higher organisms, such as plants. Even the fact that SDC is non-toxic to humans does not render it obvious that it would not be harmful to plants, which could be considered at least as closely related to fungi as they are to animals. Moreover, even if topical application to plant surfaces would not seriously damage a plant, the agent might still be harmful to the more sensitive spores or roots. Thus it was not predictable that SDC would be effective for the prevention or treatment of plant diseases while not damaging the plant itself. Further, SDC can be taken up from the soil through the plant as a systemic antimicrobial. These desirable characteristics make SDC an important new agent for controlling plant diseases.

Phakopsora pachyrhizi is an obligate parasite, meaning that it must have live, green tissue to survive. The host range of the soybean rust fungus is quite broad. In addition to soybeans, the Asian soybean rust (ASR) fungus is able to infect over 30 legumes including edible bean crops and kudzu. In November 2005, P. pachyrhizi was confirmed on Florida Beggarweed (Desmodium tortuosum) in Georgia. These additional hosts can serve as overwintering reservoirs for the pathogen and allow for build-up of inoculum, in those environs free from freezing temperatures. The pathogen is well adapted for long-distance dispersal, because spores can be readily carried long distances by the wind to new, rust-free regions. Early symptoms appear as chlorosis and brown flecking on the lower leaves in the canopy. Developing lesions can be confused with symptoms caused by other foliar diseases, such as bacterial pustule, bacterial blight, downy mildew and Septoria brown spot. The key diagnostic features of soybean rust are the cone-shaped pustules that form mostly on the undersides of the leaves and the dusty, light-tannish colored spores that erupt from the pustules. When untreated, soybean rust causes yield losses due to premature defoliation, fewer seeds per pod and decreased number of filled pods per plant.

The present disclosure can be understood more readily by reference to the following detailed description, examples, and claims, and their previous and following description. However, before the present compositions, articles, devices, and methods are disclosed and described, it is to be understood that this disclosure is not limited to the specific compositions, articles, devices, and methods disclosed unless otherwise specified, as such can, of course, vary. It is also to be understood that the terminology used herein is for the purpose of describing particular aspects only and is not intended to be limiting.

The following description of the disclosure is provided as an enabling teaching of the invention in its currently known aspects. To this end, those skilled in the relevant art will recognize and appreciate that many changes can be made to the various aspects of the disclosure described herein, while still obtaining the beneficial results of the present invention. It will also be apparent that some of the desired benefits of the present disclosure can be obtained by selecting some of the features of the present disclosure without utilizing other features. Accordingly, those who work in the art will recognize that many modifications and adaptations to the present disclosure are possible and can even be desirable in certain circumstances and are a part of the present disclosure. Thus, the following description is provided as illustrative of the principles of the present disclosure and not in limitation thereof.

Disclosed are materials, compounds, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. Thus, if a class of substituents A, B, and C are disclosed as well as a class of substituents D, E, and F and an example of a combination aspect, A-D is disclosed, then each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this disclosure including, but not limited to any components of the compositions and steps in methods of making and using the disclosed compositions. Thus, if there are a variety of additional steps that can be performed it is understood that each of these additional steps can be performed with any specific aspect or combination of aspects of the disclosed methods, and that each such combination is specifically contemplated and should be considered disclosed.

In this specification and in the claims which follow, reference will be made to a number of terms which shall be defined to have the following meanings:

As used herein, the singular forms “a,” “an” and “the” include plural referents unless the context clearly dictates otherwise. Thus, for example, reference to a “compound” includes aspects having two or more such compounds, unless the context clearly indicates otherwise.

“Optional” or “optionally” means that the subsequently described event or circumstance can or cannot occur, and that the description includes instances where the event or circumstance occurs and instances where it does not.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint.

As used herein, a “wt. %” or “weight percent” or “percent by weight” of a component, unless specifically stated to the contrary, refers to the ratio of the weight of the component to the total weight of the composition in which the component is included, expressed as a percentage.

As used herein, the term “disease” is intended to refer to any injurious or potentially injurious condition to a plant that can be caused by, for example, an organism. Exemplary diseases include, but are not limited to early leaf spot (Mycosphaerella arachidis), eyespot, Fusarium spp., powdery mildew, net blotch, phoma leaf spot, Rhynchosporium secalis, Sclerotinia sclerotiorum, Sclerotium rolfsii, Septoria tritici, Septoria nodorum, rust, tan spot, parasitic root disease, or a combination thereof. Exemplary organisms include, but are not limited to fungi, oomycetes, bacteria, viruses, viroids, virus-like organisms, phytoplasmas, protozoa, nematodes, parasitic plants, or combinations thereof. The methods and compositions of the present disclosure can be utilized on a plant afflicted with one or more diseases, or as a preventative treatment to a plant not afflicted with anyone or more diseases.

As used herein, the term “plant” is intended to refer to any member of the Kingdom Plantae, commonly referred to and recognized as plants. While not intended as a comprehensive list, commercially valuable plant susceptible to various diseases include agriculture, agronomy and horticulture species such as grasses and groundcovers, flowering plants and ornamentals, shrubs, fruit- berry- and nut-bearing trees and bushes, timber species, grains, vegetables and tuber producing species.

As used herein, the terms “agriculture” and “agricultural” can refer to crops, cultivated plants, ornamental plants, other plants and/or combinations thereof. Specifically, the term “agriculture” is intended to include, but is not limited to, horticulture, agronomy and forestry.

As used herein, the term “antimicrobial agent” is a substance capable of eliciting an antimicrobial effect, such as, for example, bacteriostatic, bacteriocidal, virucidal, virustatic, fungicidal or fungistatic.

As briefly introduced above, the present invention provides a method and composition that can be useful in, for example, treating, reducing the severity of, and/or preventing one or more diseases of a plant. The methods, in various aspects, comprise treating a plant with a composition comprising silver dihydrogen citrate. The specific methods of treatment can vary and can comprise, in various aspects, topical applications and/or systemic applications.

### Plant

The present disclosure is related to the treatment and/or prevention of one or more diseases of a plant. The plant of the present disclosure can be any plant capable of being afflicted with a disease. In various aspects, the plant can be an agricultural crop or other cultivated plant, a horticultural species, such as, for example, an ornamental plant, or a combination thereof. In a specific aspect, the plant is a crop. Exemplary crops can comprise bananas, cereals, citrus fruits, coffee, com, cotton, field beans, grapes, hops, nuts, ornamentals, peanuts, pome fruits, potatoes, rice, small fruits, soybeans, stone fruits, sugar beets, sunflowers, tea, topical fruits, turf, vegetables, and various other crops. Other exemplary plants can comprise ferns, flowering plants, conifers, and gynosperms.

In one aspect, the plant is a vascular plant, or tracheophyte, having lignified tissues for the conduction of water, nutrients, and photosynthetic products. In another aspect, the plant lacks a vascular tissue.

In yet another aspect, the plant of the present disclosure should be capable of withstanding contact with and/or exposure to the composition of the present disclosure without substantial injury.

In one aspect, the plant can be afflicted with one or more diseases. In another aspect, the plant can be free from or substantially free from affliction with one or more diseases and the methods of the present disclosure can be used, for example, to prevent and/or reduce the probability of a future disease.

### Disease

A disease of the present disclosure can be, in various aspects, any injurious or potentially injurious condition to a plant that is caused by, for example, a bacterium, a fungus a virus or other disease causing organism. Illustrative plants disease can include, in various aspects, leaf spot (Mycosphaerella arachidis), eyespot, Fusarium spp., powdery mildew, net blotch, phoma leaf spot, Rhynchosporium secalis, blight, Sclerotinia sclerotiorum, Sclerotium rolfsii, Septoria tritici, Septoria nodorum, rust, tan spot, parasitic root disease, Alternaria spp., Cercospora spp., Colletotrichum spp., Mycosphaerella spp., Pyricularia spp., Rhizoctonia solani, Septoria spp., Venturia spp. or a combination thereof. This list is not intended to be comprehensive, only illustrative. In other aspects, the disease can be anyone or more diseases afflicting a plant.

In one aspect, a disease of the present disclosure is at least partially caused by an organism. Exemplary organisms include, but are not limited to fungi, oomycetes, bacteria, viruses, viroids, virus-like organisms, phytoplasmas, protozoa, or combinations thereof. The methods and compositions of the present disclosure can be utilized on a plant susceptible to, but not afflicted with one or more diseases, or as a preventative treatment to a plant not afflicted with anyone or more diseases. It is not necessary for a plant to be afflicted with a disease to be subjected to and attain the benefits of the present invention.

### Composition

The composition of the present disclosure comprises, in various aspects, silver dihydrogen citrate, also termed SDC herein. As used herein, the term “silver dihydrogen citrate” refers to the molecule having the chemical formula AgC₆H₇0₇, along with derivatives thereof wherein a single silver cation is associated with a citrate anion. The disclosure is also intended to cover related compositions not specifically recited herein and the present disclosure is not intended to be limited to any specific formula. Silver dihydrogen citrate has been shown to have antimicrobial activity against a variety of microbes, including bacteria, fungi and viruses.

In one aspect, the composition of the present disclosure comprises silver dihydrogen citrate. The silver dihydrogen citrate can be produced from any suitable method. In one aspect, the silver dihydrogen citrate can be produced from an electrolytic process. In another aspect, the silver dihydrogen citrate can be produced from a nonelectrolytic process.

In a specific aspect, the silver dihydrogen citrate is produced by at least partially immersing an electrode comprising silver into an aqueous electrolyte solution comprising citric acid. In one aspect, the electrolyte solution comprises aqueous citric acid. In another aspect, the electrolyte solution can comprise one or more electrolytes in addition to, or instead of, citric acid. After at least partially immersing the electrode, an electrolytic potential can be applied, generating silver ions in the solution. While not wishing to be bound by theory, a silver ion can associate with and be at least partially stabilized by a citric acid molecule in solution. Such as association can produce silver dihydrogen citrate. In one aspect, the method for preparing a silver dihydrogen citrate can be the same or similar to that described in U.S. Pat. Nos. 6,197,814, 6,583,176, 6,890,953, and/or 7,261,905, which are hereby incorporated by reference in their entirety for the purpose of disclosing methods for preparing silver dihydrogen citrate.

In one aspect, a silver electrode, such as an anode, is at least about 99.9% pure Ag. In other aspects, both anode and cathode are at least about 99.9% pure Ag. In yet other aspects, pure Ag refers to about 99.99% pure Ag⁰, 99.999% pure Ag or 99.9999% pure Ag. In still other aspects, the purity of a silver electrode can be less than about 99.9% pure Ag, provided that the electrode is capable of producing silver dihydrogen citrate. In still other aspects, the anode may be made of a higher purity elemental silver (Ag) than the cathode. In one aspect, a potential difference of about 12 to 50 volts can be applied between the anode and cathode, whereby a current can flow between the two electrodes, whereby a silver ion (Ag⁺) can be released into the aqueous solution, such as, for example, citric acid.

While not wishing to be bound by theory, the greater the concentration of citric acid in solution, the greater the concentration of silver ion that can be obtainable in the solution. For example, while it has been shown that it is possible to obtain a silver ion concentration of about 0.1% Ag⁺ in a 10% aqueous citric acid solution, lower concentrations of Ag⁺ ion can be obtained using lower concentrations of citric acid, whereas higher concentrations are obtainable using higher concentrations of citric acid. It is thus possible to adjust the upper limit of the silver ion concentration in the aqueous citric acid by, for example, varying the amount of citric acid in the electrolyte solution, for example up to the maximum solubility of citric acid in water. It is likewise possible to adjust the final silver ion concentration in the aqueous citric acid, up to such upper limit, by varying the potential difference and/or the current flow between the electrodes, as well as the length of time that the voltage is applied to the electrodes while they are exposed to the electrolyte. It should be noted that the present disclosure is not limited to any particular concentration range of silver dihydrogen citrate and/or citric acid, as the limits of any method of preparing a silver dihydrogen citrate can vary depending on such factors as the purity of each component, applied potential, electrolyte concentration, temperature, or a combination thereof

In another aspect, a non-aqueous solution or a mixed solvent system can be utilized, provided that the sufficient conductivity and solubility exist to maintain an at least partially stable solution of silver dihydrogen citrate. In yet another aspect, the solution can comprise an excess of citric acid to, for example, further stabilize and buffer any produced silver dihydrogen citrate. In various aspects, the solution can comprise from 0 to about 40 wt. % of an electrolyte, such as, for example, citric acid, for example, about 0, 0.5, 1, 1.5, 2, 2.5, 3, 3.5, 4, 4.5, 5, 5.5, 6, 6.5, 7, 7.5, 8, 8.5, 9, 9.5, 10, 12, 14, 16, 18, 20, 22, 24, 26, 28, 30, 32, 34, 36, 38, or 40 wt. % citric acid. In other aspects, the solution can comprise greater than about 5 wt. % of citric acid, for example, about 5, 6, 7, 8, 9, 10, 12, 14, 16, 18, 20, 22, 24, 26, 28, 30, 32, 34, 36, 38, or 40 wt. %; or greater than about 10 wt. % of citric acid, for example, about 10, 12, 14, 16, 18, 20, 22, 24, 26, 28, 30, 32, 34, 36, 38, or 40 wt. % citric acid. In yet other aspects, the solution can comprise less than about 5 wt. % or much greater than about 10 wt. % citric acid, and the present disclosure is not intended to be limited to any particular citric acid concentration. A given amount of electrolyte, such as, for example citric acid, can be present during production of silver dihydrogen citrate and/or can be adjusted simultaneous with and/or subsequent to such production, so as to maintain a predetermined level of electrolyte and thus, at least partially control the stability of any silver dihydrogen citrate produced.

The composition of the present disclosure can be in any physical form suitable for use in the various methods of the present invention. In various aspects, the composition can comprise a spray, a foam, a liquid, a paste, a gel, a solid, a powder, a suspension, a granule, or a combination thereof. In a specific aspect, the composition comprises an aqueous solution. In another aspect, the composition comprises a solid. If, for example, the composition comprises a solid, such a solid can be produced by any suitable process. In one aspect, an electrolytically produced solution of silver dihydrogen citrate and citric acid can be dried and/or subjected to, for example, a freeze-drying process to obtain a solid material. In another aspect, the composition can comprise a granule. The specific form of any portion of the composition of the present disclosure can vary, depending upon such factors as the nature of the components, concentration of each component, and intended application of the composition.

In one aspect, the composition is in the form of a liquid. In various aspects, the liquid is aqueous and comprises silver dihydrogen citrate, water, optionally and citric acid and/or other water-soluble components, such as, for example, salts, acids, electrolytes, foaming agents, processing aids, stabilizers, anti-oxidants, and/or combinations thereof.

The composition of the present disclosure can optionally comprise a non-aqueous phase, such as an oil-phase, and/or both an aqueous and a non-aqueous phase, provided that the composition is suitable for use in delivering a silver dihydrogen citrate to a plant in accordance with the various aspects described herein.

In various aspects, the composition can be a dispersion, such as an emulsion (liquid in liquid dispersion), colloidal suspension (solid in liquid dispersion), foam (air in fluid suspension), aerosol (liquid in air dispersion), etc. In other aspects, the silver dihydrogen citrate (i.e. silver ion in aqueous organic acid) can form a continuous phase of a fluid dispersion. For example, in various aspects, the silver dihydrogen citrate can form a continuous water phase of an oil-in-water emulsion, while the dispersed oil phase can comprise one or more water-immiscible components. In such an aspect, it can be beneficial to include at least one optional emulsifier to retain, for example, droplets of the dispersed oil phase stably suspended in the continuous water phase.

In various aspects, the composition can be combined with one or more gelling agents, such as a water soluble polymer, crosslinked polymer, block copolymer or a mixture of polymers, to form, for example, a gel. A gelling agent can be a compound capable of forming a cross-linked matrix within a water solvent. In one aspect, silver dihydrogen citrate and water can fill the interstices of such a matrix. Depending on the degree of crosslinking and the amount of water used in relation to the amount of gelling agent, the resulting gel composition can have a consistency ranging from a free-flowing but viscous liquid, to a viscous fluid, to a semi-solid, to a solid of varying hardness.

### Other Composition Components

The composition of the present disclosure can also comprise one or more additional components. In one aspect, the composition can comprise an additional acid, such as, for example, acetic acid, ascorbic acid, aspartic acid, citric acid, glycolic acid, lactic acid, malic acid, malonic acid, tartaric acid, cis-cyclohexane dicarboxylic acid, chloroacetic acid, dl-cysteine, dl-cystine, propionic acid, succinic acid, or a combination thereof. In another aspect, an acid, if present can comprise one or more other acids known in the art and/or a derivative of any acid recited herein. In one aspect, anyone or more additional acids that can be present are at least partially compatible with at least one of the other components of the composition and/or the plant to be treated with the composition. In another aspect, anyone or more of the additional acids recited herein can be used in combination with, or in lieu of, any other electrolyte and/or acid.

In another aspect, the composition can comprise an additional component, such as, for example, a stabilizer, an antioxidant, a propellant, a surfactant, an emulsifier, a processing aid, a rheological aid, or a combination thereof.

In another aspect, the composition can comprise silver dihydrogen citrate alone or, in various aspects, can comprise one or more other antimicrobials and/or biocides.

In various aspects, antimicrobial preparations can be prepared by contacting and/or mixing silver dihydrogen citrate (and optionally other antimicrobial agents) with one or more other active or inert substances using customary methods, provided that such methods do not adversely affect the antimicrobial and/or biocidal activity of anyone or more active ingredients, including the silver dihydrogen citrate.

If anyone or more additional antimicrobial and/or biocide agents are present in the composition, the term “silver dihydrogen citrate,” as used herein, is distinguished from the optional other antimicrobial and/or biocidal agents which may be added to the compositions. In various aspects, the composition can comprise one or more known and/or commercially available antimicrobial and/or biocidal agents, such as, for example, an algicide, amebicide, bactericide, fungicide, germicide, viricide, or a combination thereof. In an exemplary aspect, the composition can comprise a quaternary ammonium salt. Other antimicrobial and/or biocidal agents are known and one of skill in the art could readily select an additional antimicrobial and/or biocidal agent to utilize in the composition.

The disclosure provides antimicrobial agents that can have bacteriostatic, bacteriocidal, virucidal, virustatic, fungicidal or fungistatic activities. Examples of additional antimicrobial agents that can be employed in various aspects of the present disclosure include: Pyrithiones, including the zinc complex (ZPT); Octopirox®; imethyldimethylol Hydantoin (Glydant®); Methylchloroisothiazolinone/methylisothiazolinone (Kathon CG®); Sodium Sulfite; Sodium Bisulfite; Imidazolidinyl Urea (Germall 115®, Diazolidinyl Urea (Germaill II®); Benzyl Alcohol; 2-Bromo-2-nitropropane-1,3-diol (Bronopol®); Formalin (formaldehyde); lodopropenyl Butylcarbamate (Polyphase P100®); Chloroacetamide; Methanamine; Methyldibromonitrile Glutaronitrile (1,2-Dibromo-2,4-dicyanobutane or Tektamer®); Glutaraldehyde; 5-bromo-5-nitro-1,3-dioxane (Bronidox®); Phenethyl Alcohol; o-Phenylphenollsodium o-phenylphenol; Sodium Hydroxymethylglycinate (Suttocide A®); Polymethoxy Bicyclic Oxazolidine (Nuosept C®); Dimethoxane; Thimersal; Dichlorobenzyl Alcohol; Captan; Chlorphenenesin; Dichlorophene; Chlorbutanol; Glyceryl Laurate; Halogenated Diphenyl Ethers; 2,4,4′-trichloro-2′-hydroxy-diphenyl ether (Triclosan® or TCS); 2,2′-dihydroxy-5,5′dibromo-diphenyl ether; Phenolic Compounds; Phenol; 2-Methyl Phenol; 3-Methyl Phenol; 4-Methyl Phenol; 4-Ethyl Phenol; 2,4-Dimethyl Phenol; 2,5-Dimethyl Phenol; 3,4-Dimethyl Phenol; 2,6-Dimethyl Phenol; 4-n-Propyl Phenol; 4-n-Butyl Phenol; 4-n-Amyl Phenol; 4-tert-Amyl Phenol; 4-n-Hexyl Phenol; 4-n-Heptyl Phenol; Mono- and Poly-Alkyl and Aromatic Halophenols; p-Chlorophenol; Methyl p-Chlorophenol; Ethyl p-Chlorophenol; n-Propyl p-Chlorophenol; n-Butyl p-Chlorophenol; n-Amyl p-Chlorophenol; sec-Amyl p-Chlorophenol; Cyclohexyl p-Chlorophenol; n-Heptyl p-Chlorophenol; n-Octyl p-Chlorophenol; o-Chlorophenol; Methyl o-Chlorophenol; Ethyl o-Chlorophenol; n-Propyl o-Chlorophenol; n-Butyl o-Chlorophenol; n-Amyl o-Chlorophenol; tert-Amyl o-Chlorophenol; n-Hexyl o-Chlorophenol; n-Heptyl o-Chlorophenol; o-Benzyl p-Chlorophenol; o-Benxyl-m-methyl p-Chlorophenol; o-Benzyl-m; m-dimethyl p-Chlorophenol; o-Phenylethyl p-Chlorophenol; o-Phenylethyl-m-methyl p-Chlorophenol; 3-Methyl p-Chlorophenol; 3,5-Dimethyl p-Chlorophenol; 6-Ethyl-3-methyl p-Chlorophenol; 6-n-Propyl-3-methyl p-Chlorophenol; 6-iso-Propyl-3-methyl p-Chlorophenol; 2-Ethyl-3,5-dimethyl p-Chlorophenol; 6-sec-Butyl-3-methyl p-Chlorophenol; 2-iso-Propyl-3,5-dimethyl p-Chlorophenol; 6-Diethylmethyl-3-methyl p-Chlorophenol; 6-iso-Propyl-2-ethyl-3-methyl p-Chlorophenol; 2-sec-Amyl-3,5-dimethyl p-Chlorophenol; 2-Diethylmethyl-3,5-dimethyl p-Chlorophenol; 6-sec-Octyl-3-methyl p-Chlorophenol; p-Chloro-m-cresol: p-Bromophenol; Methyl p-Bromophenol; Ethyl p-Bromophenol; n-Propyl p-Bromophenol; n-Butyl p-Bromophenol; n-Amyl p-Bromo-phenol; sec-Amyl p-Bromophenol; n-Hexyl p-Bromophenol; Cyclohexyl p-Bromophenol; o-Bromophenol; tert-Amyl o-Bromophenol; n-Hexyl o-Bromophenol; n-Propyl-m,m-Dimethyl o-Bromophenol; 2-Phenyl Phenol; 4-Chloro-2-methyl phenol; 4-Chloro-3-methyl phenol; 4-Chloro-3,5-dimethyl phenol; 2,4-Dichloro-3,5-dimethylphenol; 3,4,5,6-Terabromo-2-methyl-phenol; 5-Methyl-2-pentylphenol; 4-Isopropyl-3-methylphenol; Para-chloro-meta-xylenol (PCMX); Chlorothymol; Phenoxyethanol; Phenoxyisopropanol; 5-Chloro-2-hydroxydiphenylmethane; Resorcinol and its Derivatives; Resorcinol; Methyl Resorcinol; Ethyl Resorcinol; n-Propyl Resorcinol; n-Butyl Resorcinol; n-Amyl Resorcinol; n-Hexyl Resorcinol; n-Heptyl Re-sorcinol; n-Octyl Resorcinol; n-Nonyl Resorcinol; Phenyl Resorcinol; Benzyl Resorcinol; Phe-nylethyl Resorcinol; Phenylpropyl Resorcinol; p-Chlorobenzyl Resorcinol; 5-Chloro 2,4-Dihy-droxydiphenyl Methane; 4′-Chloro 2,4-Dihydroxydiphenyl Methane; 5-Bromo 2,4-Dihydroxy-diphenyl Methane; 4′-Bromo 2,4-Dihydroxydiphenyl Methane; Bisphenolic Compounds; 2,2′-Methylene bis-(4-chlorophenol); 2,2′-Methylene bis-(3,4,6-trichlorophenol); 2,2′-Methylene bis-(4-chloro-6-bromophenol); bis(2-hydroxy-3,5-dichlorophenyl)sulfide; bis(2-hydroxy-5-chloro-benzyl) sulfide; Benzoic Esters (Parabens); Methylparaben; Propylparaben; Butylparaben; Ethylparaben; Isopropylparaben; Isobutylparaben; Benzylparaben; Sodium Methylparaben; Sodium Propylparaben; Halogenated Carbanilides; 3,4,4′-Trichlorocarbanilides (Triclocarban® or TCC); 3-Trifluoromethyl-4,4′-dichlorocarbanilide; 3,3′,4-Trichlorocarbanilide; Chlorohexidine and its digluconate; diacetate and dihydrochloride; Undecenoic acid; Hexetidine; poly(hexamethylenebiguanide) hydrochloride (Cosmocil®).

The composition can, in various aspects, comprise non-silver antibacterial metal salts in addition to silver dihydrogen citrate. Such materials can, for example, include a salt of a metal in groups 3b-7b, 8 and 3a-5a. In various specific aspects, the composition can comprise a salt of aluminum, zirconium, zinc, gold, copper, lanthanum, tin, mercury, bismuth, selenium, strontium, scandium, yttrium, cerium, praseodymium, neodymium, promethium, samarium, europium, gadolinium, terbium, dysprosium, holmium, erbium, thulium, ytterbium, lutetium, or a combination thereof where the metal salt has antimicrobial properties.

The composition can also optionally comprise one or more chelating agents. Exemplary chelating agents are ethylene di-amine tetra acetic acid (EDTA), beta-alanine diacetic acid (EDETA), phosphonomethyl chitosan, carboxymethyl chitosan, hydroxyethylene di-amino tetraacetic acid, nitrilotriacetic acid (NTA) and ethylenediamine disuccinic acid (S,S-EDDS, R,R-EDDS or S,R-EDDS). In various aspects, a chelating agent can provide additional or synergistic effects when used in combination with silver dihydrogen citrate.

Exemplary anionic surfactants for optional use in the present disclosure are sulfated monoglycerides of the formula R₁₂—CO—O—CH₂—C(OH)H—CH₂—O—SO₃-M, wherein R₁₂ is a saturated or unsaturated, branched or unbranched alkyl group from about 8 to about 24 carbon atoms, and M is a water-soluble cation such as ammonium, sodium, potassium, magnesium, triethanolamine, diethanolamine and monoethanolamine. These are typically made by the reaction of glycerin with fatty acids (having from about 8 to about 24 carbon atoms) to form a monoglyceride and the subsequent sulfation of this monoglyceride with sulfur trioxide. An example of a sulfated monoglyceride is sodium cocomonoglyceride sulfate.

Other exemplary anionic surfactants include olefin sulfonates of the form R₁₃SO₃-M, wherein R₁₃ is a mono-olefin having from about 12 to about 24 carbon atoms, and M is a water-soluble cation such as ammonium, sodium, potassium, magnesium, triethanolamine, diethanolamine and monoethanolamine. These compounds can be produced by the sulfonation of alpha.-olefins by, for example, means of uncomplexed sulfur trioxide, followed by neutralization of the acid reaction mixture in conditions such that any sulfones which have been formed in the reaction are hydrolyzed to give the corresponding hydroxyalkanesulfonate. An example of a sulfonated olefin is sodium C₁₄/C₁₆.alpha.-olefin sulfonate.

The composition can optionally further comprise an additional proton donating agent (e.g., aside from an acid, such as, for example, citric acid, which itself can be considered a proton-donating agent). If present, an additional proton donating agent can be present in any suitable concentration, such as, for example, from about 0.1% to about 10%; from about 0.5% to about 8%; or from about 1% to about 5% (based on the weight of the composition) of a proton donating agent. A proton donating agent can be an organic acid, a polymeric acid, a mineral acid, or a mixture thereof. Such additional organic proton donating agents can be added directly to the composition in the acid form or can be formed by adding a conjugate base of the desired acid and a sufficient amount of a separate acid (for example the aforementioned organic acid) that is strong enough (i.e. has a low enough pKa) to form an undissociated acid from its conjugate base. In some embodiments, the proton donating agent can comprise a mineral acid that will not remain undissociated in the neat composition and/or when the composition is diluted during washing and rinsing. These proton donating agents can be added directly to a composition in the acid form.

In various aspects, an aqueous phase can comprise, for example, an ingredient such as an: alcohol, diol or polyol with a low number of C-atoms or their ethers (for example ethanol, isopropanol, propyleneglycol, glycerin, ethylene glycol, ethylene glycol monoethylether, ethylene glycol monobutylether, propylene glycol monomethylether, propylene glycol monoethylether, propylene glycol monobutylether, diethylene glycol monomethylether; diethylene glycol monoethylether, diethylene glycol monobutylether and similar products); a lower homolog of an alcohols (such as ethanol, isopropanol, 1,2-dipropandiol and glycerin), as well as one or more thickeners for example: silicium dioxide, aluminum silicates, polysaccharides or derivatives thereof (for example hyaluronic acid, xanthan gum, hydroxypropylmethylcellulose); polyacrylate, or a combination thereof.

In various aspects, the composition can optionally comprise an antibacterial enhancing agent, such as, for example, a polymerizable monomer, a polymer or mixture of two or more polymers such as: oligomers, homopolymers, copolymers of two or more monomers, ionomers, block copolymers, graft polymers, cross-linked polymers and copolymers, and the like. An antibacterial enhancing agent, if present, can be: natural or synthetic; water soluble or swellable (hydratable, hydrogel forming); and can have an average molecular weight of about 100 to about 5,000,000.

In other aspects, the composition can optionally comprise one or more nutrients, minerals, and/or other soluble species that can be beneficial to a plant. In a specific aspect, the composition can comprise a nutrient, such as, for example, a fertilizer and/or plant food, such that the method of treating and/or preventing a disease can optionally be combined with a routine treatment or feeding.

### Method of Treatment

The methods of the present disclosure, in various aspects, comprise contacting a composition comprising silver dihydrogen citrate with a plant to, for example, treat, reduce the severity of, and/or prevent one or more diseases of a plant. The treatment step (e.g., contacting) can comprise topically contacting the composition with a portion of the plant and/or systemically introducing the composition into the plant through the roots or any other external portion of the plant.

In various aspects, contacting the composition with the plant can result in killing and/or controlling one or more disease causing organisms, reducing the severity of a disease and/or the level of a disease causing organism related thereto, and/or preventing and/or reducing the probability of future disease formation or growth.

The concentration of anyone or more components in the composition, such as, for example, silver dihydrogen citrate, can vary, depending upon the specific components, method of contacting, severity of disease, and particular plant to be treated. In one aspect, the amount and concentration of any component in the composition if sufficient to at least partially reduce a level of disease or disease causing organisms, or to at least partially prevent a disease from occurring or growing.

In one aspect, the concentration of silver dihydrogen citrate in the composition can range from about 0.000005 wt. % to about 5 wt. % or from 0.05 ppm to about 50,000 ppm, for example, about 0.05, 0.1, 0.2, 0.5, 1, 2, 3, 4, 5, 8, 10, 12, 14, 16, 18, 20, 25, 30, 50, 75, 100, 150, 200, 300, 500, 800, 1,000, 2,000, 5,000, 8,000, 10,000, 20,000, 30,000, 40,000, or 50,000 ppm; from about 50 ppm to about 10,000 ppm; for example, about 50, 75, 100, 150, 200, 300, 500, 800, 1,000, 2,000, 5,000, 8,000, or 10,000 ppm; or from about 100 ppm to about 5,000 ppm, for example, about 100, 150, 200, 300, 500, 800, 1,000, 2,000, or 5,000 ppm. In other aspects, the concentration of silver dihydrogen citrate in the composition can be less than about 0.05 ppm or greater than about 50,000 ppm, and the present disclosure is not intended to be limited to any particular concentration range. In one aspect, the composition can be prepared and contacted with at least a portion of a plant. In another aspect, the composition can be prepared at a first concentration, for example, a concentrated solution, and subsequently diluted prior to or during contacting. Such a multistep process can be useful, for example, in transporting and/or handling a reduced volume of the composition. If such a multi-step process is used, the concentrated solution can have a silver dihydrogen citrate concentration of any value recited herein, or a greater value, provided that the silver dihydrogen citrate and any optional components that can be present, are stable or relatively stable. A diluted concentration, if used, can be any suitable concentration for contacting with a particular plant, such as, for example, from about 0.1 ppm to about 500 ppm silver dihydrogen citrate. In other aspects, a diluted concentration can be less than about 0.1 ppm or greater than about 500 ppm.

In yet other aspects, the concentration of silver dihydrogen citrate can vary depending on the specific method and rate of application. In one aspect, a portion of the composition can be applied to a single plant or to a portion thereof at a sufficient concentration to treat and/or prevent a disease. Such an application can comprise a more highly concentrated solution due to the short time duration and volume of application. In contrast, a dilute or low concentration composition can be applied to a plan by, for example, regular means, such as an irrigation system. While not wishing to be bound by theory, it is believed that a low concentration composition comprising silver dihydrogen citrate, when applied at regular intervals, can prevent the formation of a disease in a treated plant. Thus, it should be appreciated that the concentration and rate of application of a composition can vary depending upon, for example, the plants, diseases, and environmental conditions related thereto.

The composition of the present disclosure can also be a solid, such as, for example, a dry powder, a granule, or a combination thereof. If the composition comprises a solid, the concentration of silver dihydrogen citrate within the composition can vary provided that the amount of silver dihydrogen citrate is sufficient to at least partially treat and/or prevent a disease when contacted with a plant.

In one aspect, the composition of the present disclosure can be contacted with at least a portion of an external surface of a plant. Such a topical application can comprise any suitable method of contacting at any suitable concentration.

In various aspects, the composition can be contacted with a plant by spraying, such as, for example, irrigating, pouring, brushing, other suitable methods or combinations thereof.

If contacted to an external portion of a plant, the portion of the plant contacted can comprise any portion or the entirety of the plant. In various aspects, the portion of the plant can comprise a root, a stem, a wood portion, a leaf, or a combination thereof. The specific portion of a plant to be contacted can also vary depending on the type of disease and location of any diseased portion of the plant. In an exemplary aspect, a composition can be contacted with a portion of the adaxial (upper) surface of one or more leaves by, for example, spraying from above. In another exemplary aspect, a composition can be contacted with a portion of the abaxial (lower) surface of one or more leaves by, for example, spraying from below.

In another aspect, a portion of the composition can be contacted with a plant or a portion of soil and/or groundwater proximal thereto, so that at least a portion of the composition can come into direct contact with a portion of the plant and/or be at least partially absorbed into the plant by, for example, a root.

In one aspect, such a systemic application of the composition can be performed for any plant capable of absorbing a portion of the composition. In another aspect, a vascular plant, comprising xylem and phloem tissues, can be suitable for absorbing at least a portion of the composition contacted with the plant or the soil and/or groundwater proximal thereto. As used herein, the term “proximal” is intended to refer to an area adjacent to an object, such as, for example, a plant. In one aspect, proximal to a plant can refer to the area and soil and/or groundwater adjacent to the plant, such as, for example, within the root zone and/or drip zone of the plant. In another aspect, proximal to a plant can refer to an area of soil and/or groundwater such that when a composition is contacted therewith, at least a portion of the composition can be absorbed by a portion of the plant.

In one aspect, a portion of the composition can be absorbed by, for example, osmosis, at a root, such as, for example, a fine root hair. In another aspect, a portion of the composition can be transported through the plant via the xylem, together with, for example, water and/or other nutrients, such that a portion of the composition can be in dispersed in the tissues of the plant.

While not wishing to be bound by theory, it is believed that the presence of citric acid in the composition can serve to help stabilize the silver dihydrogen citrate and can also assist in the absorption of the composition by the plant.

In another aspect, a portion of the composition can be contacted directly with the soil proximal to the plant by, for example, watering, such that the composition is at least partially absorbed by the roots positioned in that portion of soil.

The presence of the composition, and specifically, the silver dihydrogen citrate disposed therein, either on an external surface of a plant or within, for example, the vascular tissues of a plant, can help treat, reduce, and/or prevent one or more diseases for the plant.

The compositions and methods of the present disclosure can have a number of benefits over conventional antimicrobial and/or biocide agents. In one aspect, the compositions and methods of the present disclosure exhibit no or substantially fewer toxicity hazards than conventional agents. In addition, the non-toxic compositions of the present disclosure can be handled without the necessity of personal protective equipment. Moreover, the compositions and method of the present disclosure are economical and environmentally friendly as compared to conventional agents.

### EXPERIMENTAL

The following examples are put forth so as to provide those of ordinary skill in the art with a complete disclosure and description of how the compositions and methods of the present disclosure are prepared, used, and evaluated, and are intended to be purely exemplary of the disclosure and are not intended to limit the scope of what the inventors regard as their disclosure. Efforts have been made to ensure accuracy with respect to numbers (e.g., amounts, temperature, etc.) but some errors and deviations should be accounted for. Unless indicated otherwise, parts are parts by weight, temperature is in ° C. (Celsius) or is at ambient temperature, and pressure is at or near atmospheric.

### Example 1

### Preparation of Silver Dihydrogen Citrate

Water was introduced into a reverse osmosis unit, and passed through a semipermeable membrane to remove impurities and produce deionized water. Anhydrous 99% pure citric acid was mixed with the water to produce 200 gallons of a 20% (wt/vol) (796 g citric acid per gallon water) solution. The 200 gallons of 20% citric acid were directed into an ion chamber containing having positive and negative electrodes, each consisting of 200 troy ounces of 999 fine silver. The positive and negative electrodes were spaced at least 2.0 mm apart, allowing the citric acid solution to pass between the two electrodes. An ion generation controller (laC) power supply including a positive and a negative conductor was attached to the positive and negative electrodes. The IGC applied a current of 5 amps at 17 volts, pulsed every 9 seconds, with a polarity change at 1 minute intervals. Throughout the process, the electrode gap was adjusted in order to maintain the 5 amp-17 volt output. The electric current flow caused an ion current to flow between the positive and negative electrodes, producing free silver ions within the diluted citric acid solution. The silver ions reacted with the citric acid in the citric acid solution to produce the silver dihydrogen citrate solution. The 20% citric acid solution was recirculated through the ion chamber at 50 gallons per minute for 144 hours until the desired silver ion concentration was obtained. The silver dihydrogen citrate solution was then allowed to sit in order to allow any solids formed during the procedure to precipitate. The resulting product was a silver dihydrogen citrate solution having a silver ion concentration of 2410 ppm.

The silver dihydrogen solution can be stored or it can be used immediately per the following examples.

It should be understood by those skilled in the art that numerous variations in the size and/or spacing of the electrodes and numerous variations in the peak voltage and numerous variations in the timing sequence of the intermittent voltage polarity can readily be used to obtain the silver dihydrogen citrate for use in the invention.

By the foregoing method, a solution was prepared having a silver ion concentration of 2410 ppm. The 2410 ppm silver ion solution was diluted in 5% aqueous citric acid, pH 7.0 to produce a silver dihydrogen citrate stock solution (stock solution) having a silver ion concentration of 100 ppm silver.

### Example 2

### Evaluation of Efficacy of SDC to Control Phakopsora pachyrhizi Spore Germination

Soybean leaves were collected which showed Asian soybean rust damage; the leaves were kept moist in order to have the maximum number of available spores. The leaves were examined with a stereoscope in order to identify those with a high degree of sporulation. Leaves were cut in standard size pieces.

Three different concentrations of SDC in distilled water were prepared having 5 ppm, 10 ppm and 15 ppm of silver, respectively. Ten ml of each solution was placed in separate test tubes. Ten selected pieces of soybean leaves were place in each test tube and stirred to suspend the spores present in the rust spots. The same procedure was used for a control, omitting the SDC in the solution. The leaves were left in the solution for two hours. For each of the product dilutions, ten Petri dishes containing water-agar substrate were inoculated with 100 spores each and maintained for 6 hours in the dark. Lactophenol dye was used to stain the mycelium growth, which was evaluated visually be microscope.

In the first experiment, approximately 16% of the spore in the control dish germinated. The results of the SDC treated spores were as follows:

| Concentration | % Germination |
|---|---|
| 5 ppm | 5.0 |
| 10 ppm | 4.0 |
| 15 ppm | 5.0 |
| Control | 16 |

These data were subjected to statistical analysis using the Skott-Knott test at the 5% probability level using SASM-Agri software, indicated that these results are significant.

A second experiment was performed using the protocol above, with the following additions. Duplicate solutions were made using Well Water from Parana State, Southern Brazil (WW) and Product Water from Reverse Osmosis (PW). The surfactant sodium lauryl sulphate was added to each solution to a concentration of 0.01% w/w. Again, duplicate Petri dishes were inoculated with 100 spores for each concentration of SDC in the two water types and for 10 the control. The results were as follows:

| Concentration | % Germination |
|---|---|
| 5 ppm WW | 1.1 |
| 5 ppm PW | 1.6 |
| 10 ppm WW | 0.5 |
| 10 ppm PW | 0.4 |
| 15 ppm WW | 0.2 |
| 15 ppm PW | 0.5 |
| Control | 40 |

The results of these experiments detailed in Example 2 indicate that under the conditions used, germination of Pakhapsora pachyrhizi spores from soybean leaves was almost totally inhibited. This inhibition was evident at concentrations of 5 ppm, 10 ppm and 15 ppm silver in distilled water, well water and product water from reverse osmosis, with or without the addition of a surfactant.

While the invention has been described with reference to the above examples, it should be understood that one of skill in the art will recognize that other embodiments can be prepared and are within the ambit of the present invention.

The references, including all United States patent documents, cited herein are incorporated herein by reference.

Throughout this application, various publications are referenced. The disclosures of these publications in their entireties are hereby incorporated by reference into this application.

It will be apparent to those skilled in the art that various modifications and variations can be made in the present invention without departing from the scope or spirit of the invention. Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

1. A method for treating a plant having or susceptible to a plant disease, the method comprising contacting at least one of the plant, a portion of soil and/or groundwater proximal to the plant, or a combination thereof with a composition comprising silver dihydrogen citrate.

2. The method of claim 1, wherein at least a portion of an external surface of the plant is contacted with said composition.

3. The method of claim 1, wherein at least one of: a root of the plant, a portion of soil and/or groundwater proximal to the plant, or a combination thereof is contacted with said composition.

4. The method of claim 3, wherein at least a portion of the composition is capable of being absorbed by the plant.

5. The method of claim 3, wherein a root of the plant is contacted with said composition.

6. The method of claim 3, wherein a portion of soil and/or groundwater proximal to the plant is contacted with said composition.

7. The method of claim 1, wherein contacting comprises an effective amount of the composition to at least reduce a level of disease.

8. The method of claim 1, wherein the composition further comprises an acid.

9. The method of claim 8, wherein the acid comprises at least one of acetic acid, ascorbic acid, aspartic acid, citric acid, glycolic acid, lactic acid, malic acid, malonic acid, tartaric acid, cis-cyclohexane dicarboxylic acid, chloroacetic acid, dl-cysteine, dl-cystine, propionic acid, succinic acid, or a combination thereof.

10. The method of claim 8, wherein the acid comprises citric acid.

11. The method of claim 1, wherein at least a portion of the composition is in the form of a spray, a foam, a liquid, a paste, a gel, a solid, a powder, a granule, or a combination thereof.

12. The method of claim 1, wherein the composition is aqueous.

13. The method of claim 1, wherein the composition is a solid.

14. The method of claim 1, wherein the composition further comprises at least one of a stabilizer, an antioxidant, a propellant, a surfactant, an emulsifier, a processing aid, a rheological aid, or a combination thereof.

15. The method of claim 1, wherein the composition further comprises at least one of an antifungal, antibacterial, antiviral agent, or a combination thereof.

16. The method of claim 1, wherein the plant is an agricultural crop, a horticultural plant, or a combination thereof.

17. The method of claim 1, wherein, after contacting, the amount of at least one of a virus, a bacteria, a fungus, a disease causing organism, or a combination thereof is at least partially reduced.
